(19) United States
(12) Patent Application Publication
(10) Pub. No.: US 2019/0275513 A1
(43) Pub. Date: Sep. 12, 2019

(54) ASPIRATING SEPARATED LIQUID COMPONENTS FROM A VESSEL

(71) Applicant: Roger S. Hogue, Maple Grove, MN (US)

(72) Inventor: Roger S. Hogue, Maple Grove, MN (US)

(21) Appl. No.: 16/295,695

(22) Filed: Mar. 7, 2019

Related U.S. Application Data
(60) Provisional application No. 62/640,013, filed on Mar. 7, 2018.

(51) Int. Cl. B01L 3/00; G01N 33/49; G01N 21/07

(52) U.S. Cl. CPC B01L 3/5021; G01N 33/491; G01N 21/07; B01L 2200/0689; B01L 2400/0487; B01L 2300/0803; B01L 2300/123; B01L 2400/0478; B01L 2300/049

(57) ABSTRACT

The disclosure includes systems and methods for aspirating liquid components from a vessel. In some system embodiments, the system includes the vessel and a diaphragm slideably coupled to a hollow inner portion of the vessel. The system may also include a seal coupled around a perimeter of the diaphragm whereby the seal is configured to seal against the hollow inner portion to thereby prevent the liquid components from passing across the diaphragm. The system may also include a flexible pipe coupled to the diaphragm such that the flexible pipe is in fluid communication with the hollow inner portion. In some embodiments, the flexible pipe extends from the diaphragm and out of the vessel.

### CROSS-REFERENCE TO RELATED APPLICATION(S)

This application claims the benefit of U.S. Provisional Application No. 62/640,013 filed on Mar. 7, 2018 for “Aspirating Separated Liquid Components From A Centrifuge Tube” by R. Hogue. U.S. Provisional Application No. 62/640,013 is hereby incorporated by reference in its entirety.

### BACKGROUND

Various embodiments disclosed herein relate to aspirating separated liquid components. More specifically, various embodiments relate to aspirating biological products such as blood, fat aspirate, bone marrow aspirate, stromal vascular fraction, and the like which have been separated into constituent components.

Isolation of platelet-rich plasma from whole blood is becoming increasingly popular for use in regenerative treatment applications across the spectrum of surgical, medical, dental, and veterinary. Platelet-rich plasma contains a myriad of 30+ growth factors, peptides, and cytokines that promote wound healing and tissue regeneration. In order to obtain a high-density concentration of platelets above physiologic baseline, an anticoagulated blood specimen must be centrifuged in order to separate whole blood into its component blood products (i.e. platelet-rich plasma, platelet-poor plasma, and red blood cells). Following centrifugation, platelet-rich plasma is separated from platelet-poor plasma in a gradient layer from the anticoagulated blood specimen, then sequestered in concentrated form through aspiration.

Conventional aspiration techniques often fail to provide a satisfactory concentration of platelets. Cross-contamination between the gradient layering of blood components is frequently encountered. Therefore, there is a need for a cost-effective device that facilitates the sequestration of platelets while minimizing cross-contamination between blood components. Additionally, there is a need for a simpler, more efficient device for aspirating separated liquid components from a vessel, such as a centrifuge tube or centrifuge bucket.

### SUMMARY

The disclosure includes systems, kits, and methods for aspirating separated liquid components from a vessel. In many embodiments, the vessel is a centrifuge tube or a centrifuge bucket.

Any aspect or embodiment of a method can be performed by a system or apparatus of another aspect or embodiment, and any aspect or embodiment of a system can be configured to perform a method of another aspect or embodiment.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of an aspiration system, according to some embodiments.

FIG. 2 illustrates a side view of a diaphragm entering a centrifuge tube, according to some embodiments.

FIGS. 3, 4, and 5 illustrate a perspective view of a diaphragm moving through a centrifuge tube to aspirate separated liquid components, according to some embodiments.

FIGS. 6 and 7 illustrate perspective views of another aspiration system, according to some embodiments.

FIG. 8 illustrates a perspective view of yet another aspiration system, according to some embodiments.

FIG. 9 illustrates a perspective view of a kit, according to some embodiments.

FIG. 10 is a perspective view of an aspiration system employing an unsealed metal centrifuge tube, according to some embodiments.

FIG. 11 is a side section view of the unsealed metal centrifuge tube shown in FIG. 10.

FIG. 12 is a perspective view of an aspiration system employing an aspiratable centrifuge bucket, according to some embodiments.

FIG. 13 is a perspective view of the aspiratable centrifuge bucket shown in FIG. 12.

FIG. 14 is a side section view of the aspiratable centrifuge bucket shown in FIG. 12.

FIGS. 15A and 15B are perspective views illustrating aspiratable centrifuge buckets mounted in a centrifuge.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The disclosure includes systems, kits, and methods for aspirating separated biologic products, including but not limited to blood products, bodily fluids, fat aspirate, bone marrow aspirate, stromal vascular fraction for use in regenerative treatment applications across the spectrum of surgical, medical, dental, and veterinary. However, it should be appreciated the embodiments disclosed herein are not limited to regenerative or medical applications. Rather, the embodiments described in this disclosure may be implemented in any application whereby liquids are separated into gradient layers.

It is an object of the present disclosure to provide a simpler, more efficient, less expensive, highly-reliable device for aspiration of liquid and/or biologic components following centrifugation and separation of liquids within a commercially-available or proprietary centrifuge tube. Additionally, it is an object of this disclosure to provide a device that is much simpler to operate than devices of the type disclosed by U.S. Pat. Nos. 7,179,391 and 7,976,796.

Embodiments disclosed herein may permit a host of chemicals, bodily fluids, and other biological products to be individually aspirated with minimal cross-contamination following centrifugation in commercially-available or proprietary centrifuge tubes. Even still, systems and methods disclosed herein may be particularly effective for sequestering a high concentration of platelet-rich plasma for use in regenerative treatments across the spectrum of surgical, medical, dental, and veterinary. As well, the teachings of this disclosure may be used effectively for aspirating a wide range of biological products, including but not limited to blood, fat aspirate, bone marrow aspirate, stromal vascular fraction, and the like which are separated into constituent components using a commercially-available or proprietary centrifuge tube.

The disclosure includes systems, kits, and methods to be used with a commercially-available or proprietary centrifuge tubes. In some embodiments, the system features a sealing diaphragm, female luer attachment on its top side with a 1-5 mm diameter tubular throughput between space above and below the sealing diaphragm, and a flexible pipe with male luer attachment on one end to attach to diaphragm and a female luer-valve attachment on the other end to attach to a syringe to allow for infusion or aspiration of fluids.

The system may be communicably engaged with the inlet/outlet port of the centrifuge tube for extending longitudinally through the tubular receptacle. After fluids to be separated by centrifugation are infused into the centrifuge tube below the sealing diaphragm through the flexible pipe connected to the sealing diaphragm's female luer attachment, residual air within the tubular receptacle immediately above the infused fluids may be aspirated from the receptacle below the diaphragm until the diaphragm makes contact with meniscus (liquid) of the infused fluids.

In some embodiments, the flexible pipe is fitted with a female luer-valve (disconnected from the syringe) at one end and a male valve (connected to diaphragm female luer fitting) at an opposite end. The flexible pipe may be flexibly coiled above the sealing diaphragm to allow a capped upper end (e.g. “cap”) of tubular receptacle to be threadably secured before centrifugation process is performed. The separated liquid components may then be introduced into and aspirated from the tubular receptacle below the diaphragm by the fluid conducting pipe attached to a syringe. As successive layers of liquid components are removed from the centrifuge tube, the sealing diaphragm may descend through the tubular receptacle as the flexible aspiration pipe is extended and descends in contact with the descending meniscus (liquid).

### First System Embodiments

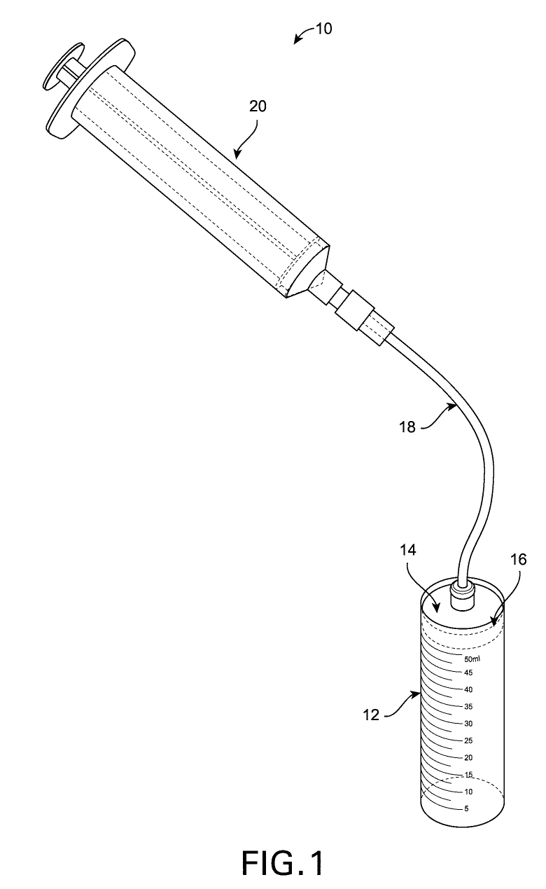

Regarding specific embodiments, as shown in FIG. 1, the disclosure includes a system 10 for aspirating liquid components from a tube 12. The system 10 may thereby include the tube 12, which includes a hollow inner portion, a sidewall radially extending around the hollow inner portion, an open end, and a closed end opposite the open end.

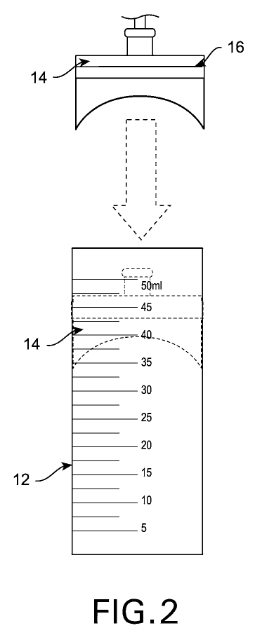

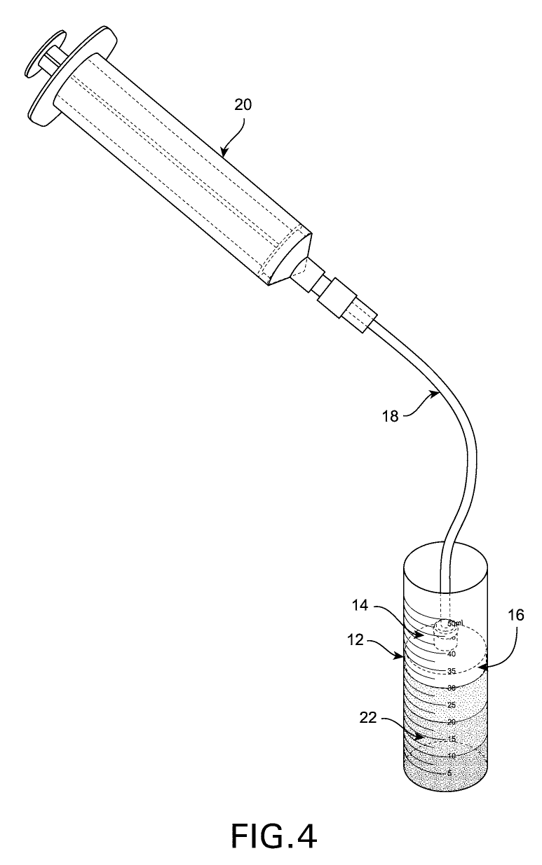

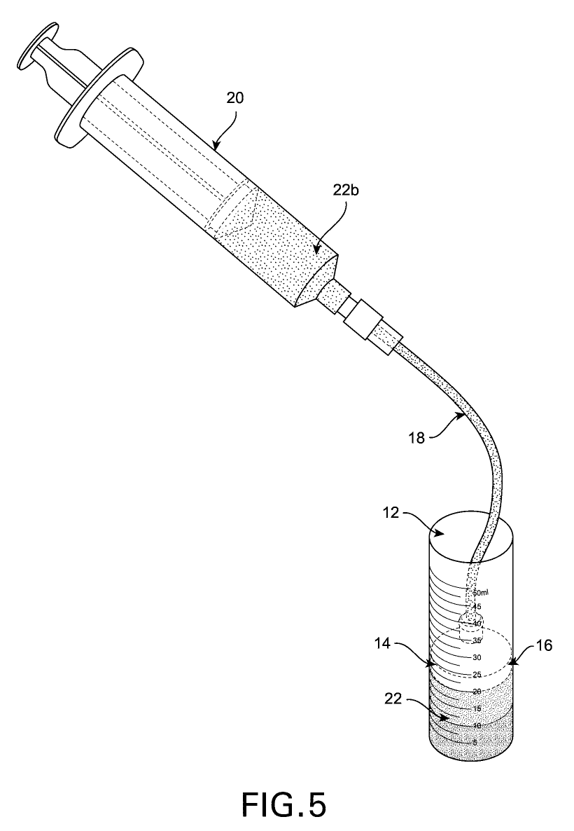

The system 10 may also include a diaphragm 14 slideably coupled to the hollow inner portion of the tube 12. As illustrated in FIGS. 2-5, the diaphragm 14 may be configured to move towards the closed end and the open end within the tube 12. In some embodiments, a first volume of the hollow inner portion located between the closed end and the diaphragm 14 defines a first portion and a second volume of the hollow inner portion located between the diaphragm 14 and the open end defines a second portion. The diaphragm 14 may also comprise an aperture that is in fluid communication with the first portion. In some embodiments, the aperture may define a diameter greater than or equal to 1 millimeter (“mm”) and less than or equal to 5 mm. However, it should be appreciated that the aperture diameter may define any diameter greater than or equal to 5 mm and less than or equal to 1 mm.

In many embodiments, the system 10 includes a seal 16 coupled around a perimeter (“outer surface”) of the diaphragm 14. The seal 16 may be configured to seal the diaphragm 14 against the hollow inner portion of the tube 12 to thereby prevent the liquid components from passing along the outer surface of the diaphragm 14 from the first portion of the tube 12 to the second portion of the tube 12. The seal 16 may be located at any vertical location along the perimeter of the diaphragm 14, such as towards the bottom portion of the diaphragm 14, the top portion of the diaphragm 14, or any location in between. However, it should be appreciated that the system 10 does not require a seal 16 and the system 10 may be unsealed. In other words, the diaphragm 14, when inserted into the tube 12, may not seal the first portion of the tube 12 from the second portion of the tube 12.

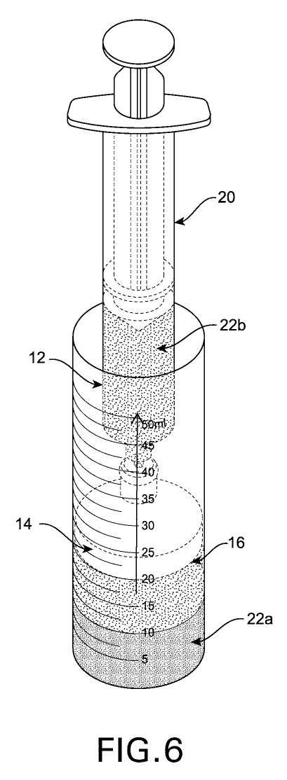

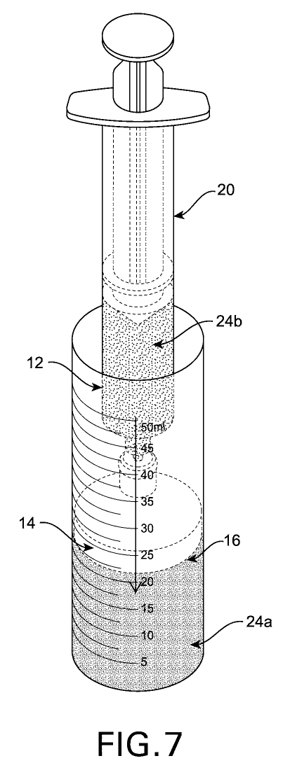

The system 10 may also include a flexible pipe 18 having a first open end and a second open end located opposite the first open end. The first open end may be coupled to the diaphragm 14 such that the flexible pipe 18 is in fluid communication with the first portion. The flexible pipe 18 may thereby extend from the diaphragm 14 into the second portion and out of the tube 12. However, as illustrated in FIGS. 6 and 7, the system 10 does not require a flexible pipe 18, instead a syringe 20 may be directly coupled to the diaphragm 14.

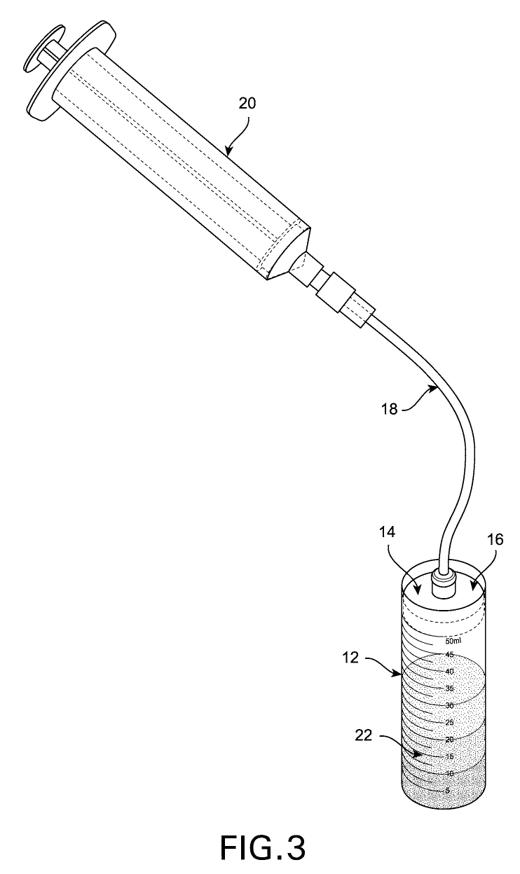

As previously stated, embodiments described in this disclosure may be implemented in many different applications from medical to any non-medical application. However, all applications are used to aspirate liquid from a tube 12. As shown in FIGS. 3-5, in many embodiments, the liquid components 24 are separated liquid components 22 and the tube 12 is a centrifuge tube.

The tube 12 may be configured in a variety of shapes and sizes. In some embodiments, the hollow inner portion of the tube 12 tapers inward from the open end to the closed end. While in some embodiments, the hollow portion of the tube 12 defines an inner tube diameter that is substantially the same from the open end to the closed end. In other words, the hollow inner portion of the tube 12 does not taper and remains substantially flat (“straight”) from the open end to the closed end. Even still, in some embodiments, the hollow inner portion of the tube 12 tapers outward from the open end to the closed end. Additionally, the closed end of the tube 12 may define various shapes, such as a conical shape, a flat shape, a rounded shape, and the like. Moreover, the centrifuge tube may define a volume substantially equal to 15 milliliters (“ml”), 50 ml, 100 ml, and any size greater than, smaller than, or in between 15 ml, 50 ml, and 100 ml.

As further shown in FIGS. 1 and 3-7, some embodiments include a syringe 20 coupled to the second open end of the flexible pipe 18. It should be appreciated that the syringe 20 is configured to receive at least a portion of the separated liquid components 22. The syringe 20 may also be used to inject liquid components 24 from the syringe 20 through the diaphragm 14 and into the tube 12.

Because one of the purposes of the present disclosure and accompanying embodiments is to provide a lower cost solution, some embodiments may be configured with commercially available components. For example, the diaphragm 14 may include a female luer attachment coupled to a top portion of the diaphragm 14. The female luer attachment may be configured to receive the first open end of the flexible pipe 18. Accordingly, the first open end of the flexible pipe 18 may include a male luer attachment configured to couple to the female luer attachment along the top portion of the diaphragm 14. In a specific embodiment, the flexible pipe 18 may include at its first open end a swabbable needleless female luer connector that serves to provide a seal and a closed-system environment with the diaphragm 14 and the liquid components in the hollow interior of the tube 12. For example, a commercially available CARESITE® Smallbore Extension Set (REF 470101) may be used in some embodiments. The second open end of the flexible pipe 18 may include a female luer-valve attachment configured to couple to the syringe 20. The female luer-valve attachment may be configured to control the flow of air and liquid from the tube 12 into the syringe 20, and conversely from the syringe 20 into the tube 12.

The seal 16 may be biocompatible with whole blood, fat aspirate, and bone marrow aspirate. In some embodiments, the seal 16 comprises silicone. For non-medical applications, the seal 16 may comprise any type of material that is configured to seal against a surface, such as a tubular surface.

As illustrated in FIG. 2, the diaphragm 14 may define a variety of shapes and sizes that are sized and configured to effectively aspirate liquid from the tube 12 into the syringe 20, via the flexible pipe 18. As shown in FIG. 2, a bottom side of the diaphragm 14 may define a convex shape. Additionally, the bottom side of the diaphragm 14 may define a concave shape located within the convex shape. However, it should be appreciated that the bottom side of the diaphragm 14 may define any shape, such as a convex shape, concave shape, triangular shape, flat shape, and the like.

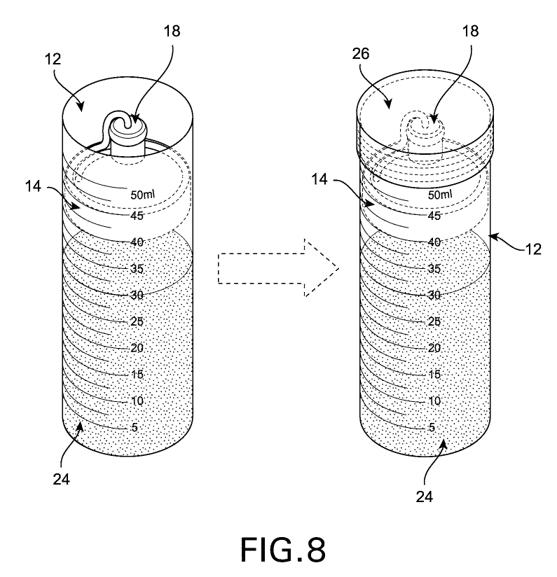

Now, with reference to FIG. 8, the system may further include a cap 26 coupled to the open end of the tube 12. The cap 26 may be configured to enclose and seal the hollow inner portion of the tube 12 for centrifugation of the liquid components 24 within the tube 12. As further shown in FIG. 8, the flexible pipe 18 may be coiled up within the second portion. In this regard, the diaphragm 14 and flexible pipe 18 may be inserted into the hollow portion of the tube 12 after centrifugation or before centrifugation whereby the diaphragm 14 and flexible pipe 18 would undergo the centrifugation process. The cap 26 may thereby be coupled to the open end of the tube 12 to thereby enclose the diaphragm 14 and flexible pipe 18 within the second portion of the tube 12 to seal the contents for centrifugation.

### First Kit Embodiments

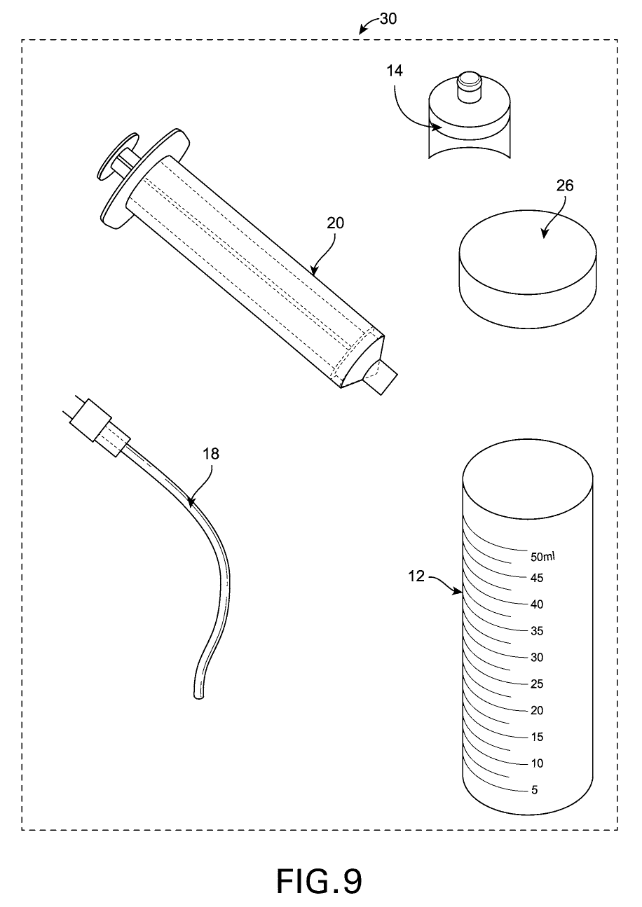

As shown in FIG. 9, the disclosure also includes a kit 30 for aspirating liquid components from a tube 12, whereby the kit 30 comprises any and all of the components as described above. In this regard, the kit 30 may include commercially available components that are sold to practitioners and consumers as a ready-to-assemble and use kit. For example, the kit 30 may include a tube 12 having a hollow inner portion, a sidewall radially extending around the hollow inner portion, an open end, and a closed end opposite the open end.

The kit may also include a diaphragm 14 sized and configured to slide within the hollow inner portion towards the closed end and the open end. The diaphragm 14 may comprise an aperture that extends through the diaphragm 14. Furthermore, the diaphragm 14 may comprise a seal 16 coupled around a perimeter of the diaphragm 14. As previously described, the seal 16 may be sized and configured to seal against the hollow inner portion to thereby prevent liquid components 24 from leaking through the diaphragm 14 and seal 16.

Additionally, the kit 30 may include a flexible pipe 18 having a first open end and a second open end located opposite the first open end. As previously described, the flexible pipe 18 may be used to remove air from the hollow inner portion of the tube and thereby aspirate liquid components 24 and separated liquid components 22 from the hollow inner portion.

The kit 30 may also include a cap 26 configured to couple to the open end of the tube 12 to thereby enclose and seal the hollow inner portion of the tube 12. As well, the kit 30 may include a syringe 20 configured to couple to the second open end of the flexible pipe 18.

As previously stated and now reiterated, the kit 30 may include any components recited above in regards to the system 10. Additionally, the kit 30 may include any different combination of components. For example, in some embodiments the kit 30 comprises a tube 12, diaphragm 14, and seal 16. Generally, it should be appreciated that the kit 30 may include any combination of the following six components: tube 12, diaphragm 14, seal 16, flexible pipe 18, syringe 20, and cap 26.

### First Method Embodiments

The disclosure also includes methods of using the system 10 and kit 30 as described above. The methods disclosed may provide a variety of sequences for aspirating the liquid components 24 from the tube 12. In some embodiments, the diaphragm 14 and the flexible pipe 18 are inserted into the hollow portion of the tube 12 before injecting the liquid components 24 into the tube 12 and of course, before centrifugation of the liquid components 24. In this regard, the liquid components 24 may be injected from the syringe 20 through the diaphragm 14 and flexible pipe 18 and into the tube 12. As such, the diaphragm 14 and flexible pipe 18 may serve as an inlet and outlet for the liquid components 24 to be injected into the tube 12 or for separated liquid components 22 to be aspirated out of the tube 12 and into a secondary device, such as the syringe 20.

In some embodiments, the diaphragm 14 and the flexible pipe 18 are inserted into the hollow portion of the tube 12 after injecting the liquid components 24 into the tube 12 but before centrifugation of the liquid components 24. Even still, in some embodiments, the diaphragm 14 and the flexible pipe 18 may be inserted into the hollow portion of the tube 12 after injecting the liquid components 24 into the tube 12 and after centrifugation of the liquid components 24. As such, while the diaphragm 14 and flexible pipe 18 may serve as an inlet and outlet in such methods, the diaphragm 14 and flexible pipe 18 may only need to serve as an outlet for the liquid components 24 to be aspirated out of the tube 12. Generally, the diaphragm 14 and flexible pipe 18 may serve as an inlet and/or outlet and the diaphragm 14 and the flexible pipe 18 may be added after centrifugation or at any point before centrifugation.

As shown in FIG. 7, some methods may include injecting liquid into the tube 12 having a hollow inner portion, a sidewall radially extending around the hollow inner portion, an open end, and a closed end opposite the open end, wherein the liquid comprises the liquid components 24. Methods may include separating the liquid into gradient layers within the tube 12 and inserting a diaphragm 14 into the hollow inner portion. As previously stated, the steps of separating the liquid into gradient layers and inserting the diaphragm 14 into the hollow portion may occur in any such sequential order.

As shown in FIGS. 3-5, methods may also include moving the diaphragm 14 towards the closed end of the tube 12. As such, methods may also include aspirating a first layer of liquid from the tube 12 through at least one of the diaphragm 14 and the flexible pipe 18. Additionally, methods may include sealing the first portion from the second portion via a seal 16 that extends around a perimeter of the diaphragm 14 to thereby prevent the liquid components 24 from passing from the first portion to the second portion.

Some embodiments may include a flexible pipe 18 that extends from the diaphragm 14, such methods may include coiling the flexible pipe 18 and inserting the flexible pipe 18 into the second portion of the tube 12. Methods may also include coupling a cap 26 to the open end of the tube 12 to thereby enclose the first portion and the second portion from an outside environment. Coupling the cap 26 to the open end of the tube 12 may comprise threadably coupling the cap 26 to the open end of the tube 12. It should be appreciated that the cap 26 may be coupled to the tube 12 under any scenario, such as when a diaphragm 14 and accompanying flexible pipe 18 are located within the tube 12, when a diaphragm 14 and no flexible pipe 18 are located within the tube 12, and when neither a diaphragm 14 and flexible pipe 18 are located within the tube 12.

### Second System Embodiments

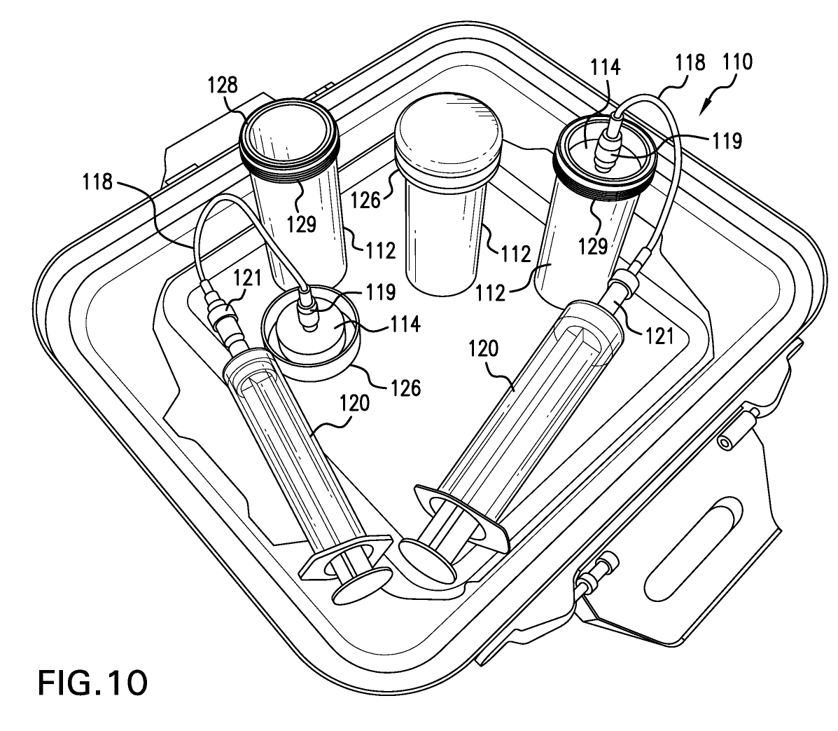

In a second variation of system embodiments, as shown in the perspective view of FIG. 10, the disclosure includes a system 110 for aspirating liquid components from a tube 112. The system 110 may thereby include the tube 112, which includes a hollow inner portion, a sidewall radially extending around the hollow inner portion, an open end, and a closed end opposite the open end. As shown in FIG. 10, the tube 112 may be composed of metal, such as stainless steel, in some embodiments.

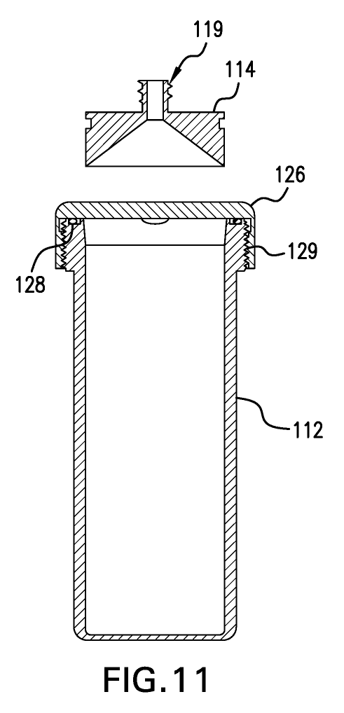

The system 110 also includes a diaphragm 114 slideably coupled to the hollow inner portion of the tube 112. FIG. 11 is a side section view that illustrates the tube 112 and the diaphragm 114 in more detail. The diaphragm 114 may have a bottom surface in the example shown that is conically shaped, with the peak of the cone facing the top at the aperture of the diaphragm 114. The diaphragm 114 may be configured to move towards the closed end and the open end within the tube 112. In some embodiments, a first volume of the hollow inner portion located between the closed end and the diaphragm 114 defines a first portion and a second volume of the hollow inner portion located between the diaphragm 114 and the open end defines a second portion. The diaphragm 114 may also comprise an aperture that is in fluid communication with the first portion. In some embodiments, the aperture may define a diameter greater than or equal to 1 millimeter (“mm”) and less than or equal to 5 mm. However, it should be appreciated that the aperture diameter may define any diameter greater than or equal to 5 mm and less than or equal to 1 mm.

In many embodiments, the diaphragm 114 is configured so that its perimeter (“outer surface”) is very closely fit against the sidewall of the hollow inner portion of the tube 112. This close fit does not provide an air-tight or liquid-tight seal between the outer surface of the diaphragm 114 and the sidewall of the hollow inner portion of the tube 112, but generally prevents movement of the diaphragm 114 within the tube 112, such that movement of the diaphragm 114 is effected via air and/or liquid suction/inflow by a syringe 120 connected to the diaphragm 114.

The system 110 may also include a flexible pipe 118 having a first open end and a second open end located opposite the first open end. The first open end may be coupled to the diaphragm 114 by an interface 119 (such as a luer-lock system in some embodiments, described in more detail below), such that the flexible pipe 118 is in fluid communication with the first portion. The flexible pipe 118 may thereby extend from the diaphragm 114 into the second portion and out of the tube 112, with the opposite end of the flexible pipe 118 being connected to a syringe 120 by an interface 121 (such as a luer-lock system in some embodiments, described in more detail below). In some embodiments, the system 110 does not require a flexible pipe 118, instead a syringe 120 may be directly coupled to the diaphragm 114.

As previously stated, embodiments described in this disclosure may be implemented in many different applications from medical to any non-medical application. However, all applications are used to aspirate liquid from a tube 112. Similar to the illustrations of the first system embodiments in FIGS. 3-5, in many applications, liquids are separated into various liquid components, and the tube 112 is a centrifuge tube.

The tube 112 may be configured in a variety of shapes and sizes. In some embodiments, the hollow inner portion of the tube 112 tapers inward from the open end to the closed end. In other embodiments, the hollow portion of the tube 112 defines an inner tube diameter that is substantially the same from the open end to the closed end. In other words, the hollow inner portion of the tube 112 does not taper and remains substantially flat (“straight”) from the open end to the closed end. Even still, in some embodiments, the hollow inner portion of the tube 112 tapers outward from the open end to the closed end. Additionally, the closed end of the tube 112 may define various shapes, such as a conical shape, a flat shape, a rounded shape, and the like. Moreover, the centrifuge tube may define a volume substantially equal to 15 milliliters (“ml”), 50 ml, 100 ml, and any size greater than, smaller than, or in between 15 ml, 50 ml, and 100 ml.

As shown in FIG. 10, a syringe 120 is coupleable to the second open end of the flexible pipe 118 by interface 121. It should be appreciated that the syringe 120 is configured to receive at least a portion of the separated liquid components contained in tube 112. The syringe 120 may also be used to inject liquid components from the syringe 120 through the diaphragm 114 and into the tube 112.

In some examples, the interface 119 between the diaphragm 114 and the flexible pipe 118 may include a female luer attachment coupled to a top portion of the diaphragm 114. The female luer attachment may be configured to receive the first open end of the flexible pipe 118. Accordingly, the first open end of the flexible pipe 118 may include a male luer attachment configured to couple to the female luer attachment along the top portion of the diaphragm 114. In a specific embodiment, the flexible pipe 118 may include at its first open end a swabbable needleless female luer connector that serves to provide a seal and a closed-system environment with the diaphragm 114 and the liquid components in the hollow interior of the tube 112. For example, a commercially available CARESITE® Smallbore Extension Set (REF 470101) may be used in some embodiments. The second open end of the flexible pipe 118 may include a female luer-valve attachment configured to couple to the syringe 120 at interface 121. The female luer-valve attachment may be configured to control the flow of air and liquid from the tube 112 into the syringe 120, and conversely from the syringe 120 into the tube 112.

The tube 112 has an open end at its top. However, in many embodiments, when liquid or other material is added to the tube 112, such as for spinning in a centrifuge in the case where tube 112 is a centrifuge tube, a top seal over the open end of the tube 112 is needed. The cap 126 is configured to provide a top seal to the tube 112, in cooperation with an O-ring 128, and is configured to be threadedly secured to the tube 112 via threads 129 on the tube 112 and complementary threads on the inside rim of the cap 126. In the illustration of FIG. 10, the tube 112 on the left side of the drawing has the O-ring 128 in place on the top rim of tube 112, while the cap 126 is off (and is laying upside-down in the tray with diaphragm 114 sitting on it to maintain sterility in a non-deployed state). The tube 112 in the center of the drawing in FIG. 10 has the cap 126 secured to the tube 112, thereby sealing the hollow inner portion of the tube 112.

### Second Method Embodiments

The disclosure also includes methods of using the system 110 as described above. The methods disclosed may provide a variety of sequences for aspirating liquid components from the tube 112. First, liquid material is added to the tube 112, such as by injection with a syringe, or by simply pouring the liquid from a container into the tube 112 through the top open end thereof. Once the liquid material is in the tube 112, the cap 126 is secured via the threads 129 to the tube 112, with the O-ring 128 in place to help ensure that the tube 112 is sealed. The tube 112 (along with one or more additional tubes 112 that also contain liquid material, in some embodiments) is then placed into a centrifuge bucket of a centrifuge, for performance of a spinning process that separates the liquid material in the tube into different, separated liquid components.

After separation in the centrifuge, the cap 126 (and possibly (separately) the O-ring 128) is removed from the tube 112, and the diaphragm 114, with the flexible pipe 118 connected thereto, may be inserted into the hollow portion of the tube 112. Together, the diaphragm 114 and the flexible pipe 118 (under control of the syringe 120) serve as an outlet for the liquid components to be aspirated out of the tube 112. Specifically, once the diaphragm 114 is inserted into the tube 112, with the flexible pipe 118 connected to the diaphragm 114, the syringe 120 is connected to the opposite end of the flexible pipe 118, and the syringe is operated to aspirate air that is contained in the top-most portion of the tube 112. This process will cause the diaphragm 114 to move downward toward the bottom closed end of the tube 112. Once all of the air has been aspirated from the tube 112 into the syringe 120, the syringe 120 is disconnected from the flexible pipe 118 at interface 121, and the aspirated air is expelled from the syringe 120. Next, the syringe is re-connected to the flexible pipe 118 at interface 121, and the next volume of liquid components is aspirated into the syringe 120. In one example, this volume of liquid components may be made up of plasma that has been separated from blood. This process may continue until all of the different liquid components in the tube 112 have been separately aspirated into the syringe 120, and possibly expelled into an appropriate container or other location.

### Third System Embodiments

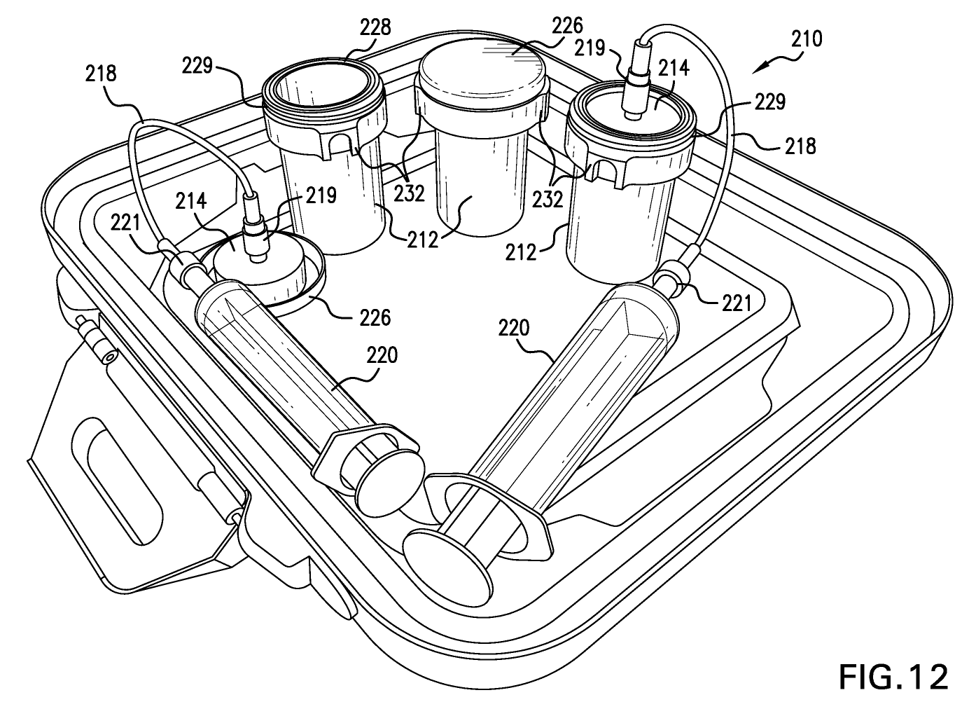

In a third variation of system embodiments, as shown in the perspective view of FIG. 12, the disclosure includes a system 210 for aspirating liquid components from a hybrid centrifuge tube/bucket 212 (referred to as “bucket 212” hereafter). The system 210 may thereby include the bucket 212, which includes a hollow inner portion, a sidewall radially extending around the hollow inner portion, an open end, and a closed end opposite the open end. As shown in FIG. 12, the bucket 212 may be composed of metal, such as stainless steel, in some embodiments.

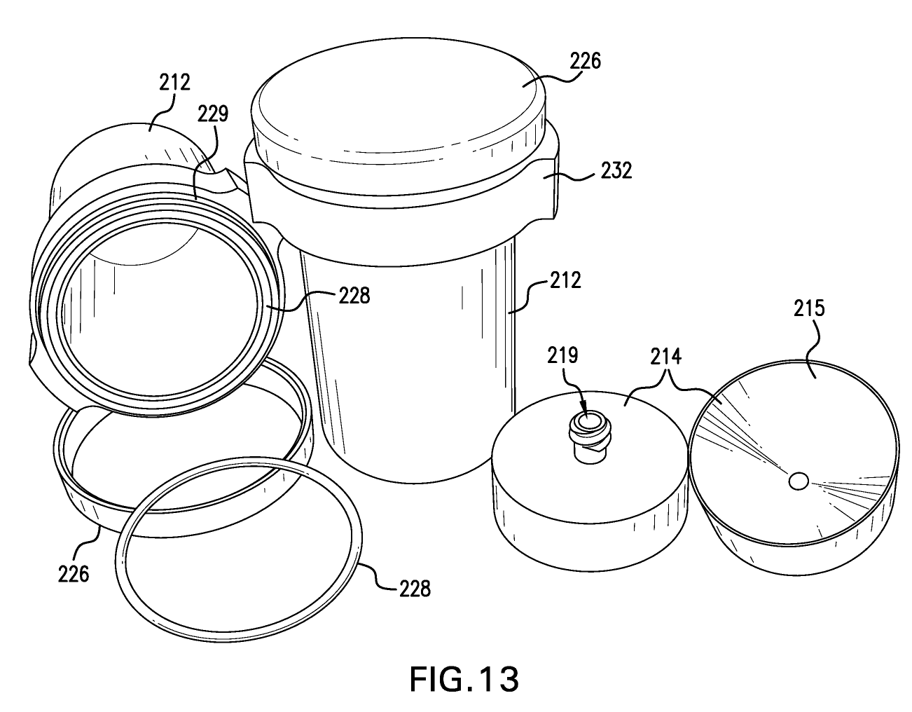

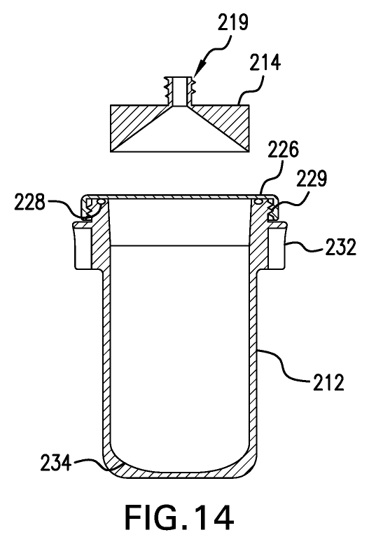

The system 210 also includes a diaphragm 214 slideably coupled to the hollow inner portion of the bucket 212. FIG. 13 is a perspective view of the bucket 212 with various components (such as the diaphragm 214) removed and shown separately, and FIG. 14 is a side section view that illustrates the bucket 212 and the diaphragm 214 in more detail. The diaphragm 214 has a bottom surface 215 in the example shown that is conically shaped, with the peak of the cone facing the top at the aperture of the diaphragm 214. The diaphragm 214 may be configured to move towards the closed end and the open end within the bucket 212. In some embodiments, a first volume of the hollow inner portion located between the closed end and the diaphragm 214 defines a first portion and a second volume of the hollow inner portion located between the diaphragm 214 and the open end defines a second portion. The diaphragm 214 may also comprise an aperture that is in fluid communication with the first portion. In some embodiments, the aperture may define a diameter greater than or equal to 1 millimeter (“mm”) and less than or equal to 5 mm. However, it should be appreciated that the aperture diameter may define any diameter greater than or equal to 5 mm and less than or equal to 1 mm.

In many embodiments, the diaphragm 214 is configured so that its perimeter (“outer surface”) is very closely fit against the sidewall of the hollow inner portion of the bucket 212. This close fit does not provide an air-tight or liquid-tight seal between the outer surface of the diaphragm 214 and the sidewall of the hollow inner portion of the bucket 212, but generally prevents movement of the diaphragm 214 within the bucket 212, such that movement of the diaphragm 214 is effected via air and/or liquid suction/inflow by a syringe 220 connected to the diaphragm 214.

The system 210 may also include a flexible pipe 218 having a first open end and a second open end located opposite the first open end. The first open end may be coupled to the diaphragm 214 by an interface 219 (such as a luer-lock system in some embodiments, described in more detail below), such that the flexible pipe 218 is in fluid communication with the first portion. The flexible pipe 218 may thereby extend from the diaphragm 214 into the second portion and out of the bucket 212, with the opposite end of the flexible pipe 218 being connected to a syringe 220 by an interface 221 (such as a luer-lock system in some embodiments, described in more detail below). In some embodiments, the system 210 does not require a flexible pipe 218, instead a syringe 220 may be directly coupled to the diaphragm 214.

As previously stated, embodiments described in this disclosure may be implemented in many different applications from medical to any non-medical application. However, all applications are used to aspirate liquid from a vessel. Similar to the illustrations of the first system embodiments in FIGS. 3-5, in many applications, liquids are separated into various liquid components, and the bucket 212 is used in place of a standard centrifuge bucket that is ordinarily configured to receive and hold a centrifuge tube.

The bucket 212 may be configured in a variety of shapes and sizes, although the configuration of the bucket 212 is partially defined by the design of the centrifuge in which the bucket 212 will be used. In some embodiments, the hollow inner portion of the bucket 212 tapers inward from the open end to the closed end. In other embodiments, the hollow portion of the bucket 212 defines an inner diameter that is substantially the same from the open end to the closed end. In other words, the hollow inner portion of the bucket 212 does not taper and remains substantially flat (“straight”) from the open end to the closed end. Even still, in some embodiments, the hollow inner portion of the bucket 212 tapers outward from the open end to the closed end. Additionally, the closed end of the bucket 212 may define various shapes, such as a conical shape, a flat shape, a rounded shape, and the like. In the example shown in FIG. 14, the closed end of the bucket has a rounded/conical shape 234. Moreover, the bucket 212 may define a volume substantially equal to 15 milliliters (“ml”), 50 ml, 100 ml, and any size greater than, smaller than, or in between 15 ml, 50 ml, and 100 ml.

As shown in FIG. 12, a syringe 220 is coupleable to the second open end of the flexible pipe 218 by interface 221. It should be appreciated that the syringe 220 is configured to receive at least a portion of the separated liquid components contained in bucket 212. The syringe 220 may also be used to inject liquid components from the syringe 220 through the diaphragm 214 and into the bucket 212.

In some examples, the interface 219 between the diaphragm 214 and the flexible pipe 218 may include a female luer attachment coupled to a top portion of the diaphragm 214. The female luer attachment may be configured to receive the first open end of the flexible pipe 218. Accordingly, the first open end of the flexible pipe 218 may include a male luer attachment configured to couple to the female luer attachment along the top portion of the diaphragm 214. In a specific embodiment, the flexible pipe 218 may include at its first open end a swabbable needleless female luer connector that serves to provide a seal and a closed-system environment with the diaphragm 214 and the liquid components in the hollow interior of the bucket 212. For example, a commercially available CARESITE® Smallbore Extension Set (REF 470101) may be used in some embodiments. The second open end of the flexible pipe 218 may include a female luer-valve attachment configured to couple to the syringe 220 at interface 221. The female luer-valve attachment may be configured to control the flow of air and liquid from the bucket 212 into the syringe 220, and conversely from the syringe 220 into the bucket 212.

The bucket 212 has an open end at its top. However, in many embodiments, when liquid or other material is added to the bucket 212, such as for spinning in a centrifuge, a top seal over the open end of the bucket 212 is needed. The cap 226 is configured to provide a top seal to the bucket 212, in cooperation with an O-ring 228, and is configured to be threadedly secured to the bucket 212 via threads 129 on the bucket 212 and complementary threads on the inside rim of the cap 226. In the illustration of FIG. 12, the bucket 212 on the left side of the drawing has the O-ring 228 in place on the top rim of bucket 212, while the cap 226 is off (and is laying upside-down in the tray with diaphragm 214 sitting on it to maintain sterility in a non-deployed state). The bucket 212 in the center of the drawing in FIG. 12 has the cap 226 secured to the bucket 212, thereby sealing the hollow inner portion of the bucket 212.

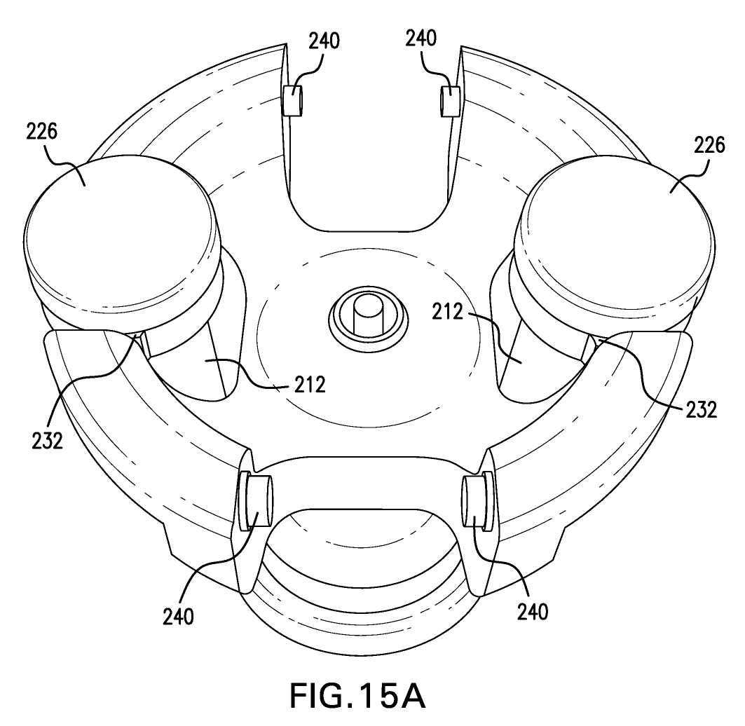

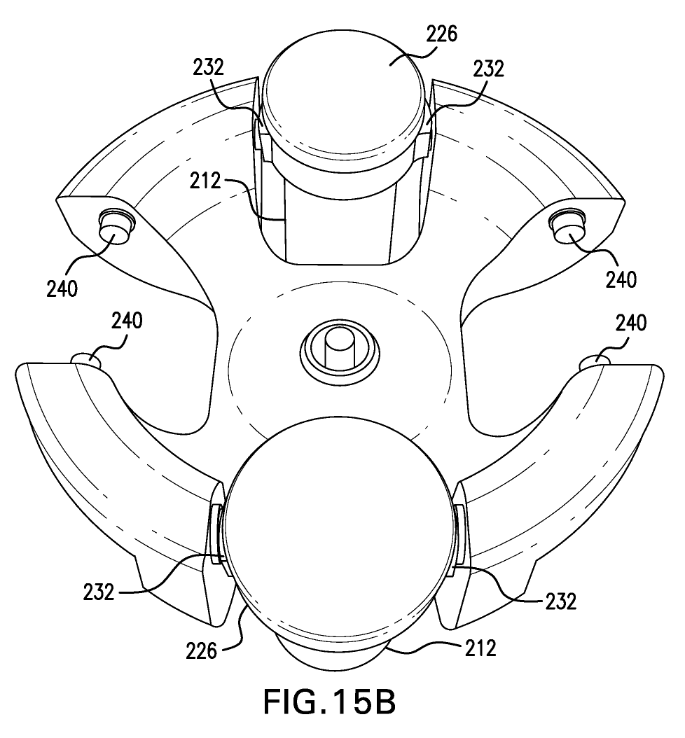

The bucket 212 includes brackets 232 near the top open end thereof, which are configured to receive pins 240 of a centrifuge (see FIGS. 15A and 15B) to allow the bucket 212 to be secured in place in the centrifuge in the customary “hanging” manner for buckets of the centrifuge. This will be described in more detail in the description below of methods of using the bucket 212 in a centrifuge.

### Third Method Embodiments

The disclosure also includes methods of using the system 210 as described above. The methods disclosed may provide a variety of sequences for aspirating liquid components from the bucket 212. First, liquid material is added to the bucket 212, such as by injection with a syringe, or by simply pouring the liquid from a container into the bucket 212 through the top open end thereof. Once the liquid material is in the bucket 212, the cap 226 is secured via the threads 229 to the bucket 212, with the O-ring 228 in place to help ensure that the bucket 212 is sealed. The bucket 212 (along with one or more additional buckets 212 that also contain liquid material, in some embodiments) is then loaded into a centrifuge, for performance of a spinning process that separates the liquid material in the bucket 212 into different, separated liquid components. FIGS. 15A and 15B illustrate an example in which two buckets 212 are loaded into an Eppendorf 5702 centrifuge system (although other centrifuge systems may be used, with corresponding variations in the sizes and mounting configurations of the buckets 212 to be compatible with the centrifuge system). In this example, the buckets 212 are mounted with the brackets 232 receiving the pins 240 in each of the centrifuge stalls, to allow the buckets 212 to be secured in place in the centrifuge in the customary “hanging” manner for buckets of the centrifuge. FIG. 15A illustrates the buckets 212 in a first position (at 3 o'clock and 9 o'clock), and FIG. 15B illustrates the buckets 212 in a second position (at 6 o'clock and 12 o'clock) after a partial rotation by the centrifuge.

After separation in the centrifuge, the cap 226 (and possibly (separately) the O-ring 228) is removed from the bucket 212, and the diaphragm 214, with the flexible pipe 218 connected thereto, may be inserted into the hollow portion of the bucket 212. Together, the diaphragm 214 and the flexible pipe 218 (under control of the syringe 220) serve as an outlet for the liquid components to be aspirated out of the bucket 212. Specifically, once the diaphragm 214 is inserted into the bucket 212, with the flexible pipe 218 connected to the diaphragm 214, the syringe 220 is connected to the opposite end of the flexible pipe 218, and the syringe is operated to aspirate air that is contained in the top-most portion of the bucket 212. This process will cause the diaphragm 214 to move downward toward the bottom closed end of the bucket 212. Once all of the air has been aspirated from the bucket 212 into the syringe 220, the syringe 220 is disconnected from the flexible pipe 218 at interface 221, and the aspirated air is expelled from the syringe 220. Next, the syringe is re-connected to the flexible pipe 218 at interface 221, and the next volume of liquid components is aspirated into the syringe 220. In one example, this volume of liquid components may be made up of plasma that has been separated from blood. This process may continue until all of the different liquid components in the bucket 212 have been separately aspirated into the syringe 220, and possibly expelled into an appropriate container or other location.

### Fourth Method Embodiments

In conjunction with any of the method embodiments discussed above, the disclosure may include a further method of aspirating separated liquid components. In some particular embodiments, this further method may follow the steps of separating liquid components in a vessel (tube 12, tube 112 or bucket 212), inserting a diaphragm (14, 114, 214) into the vessel, and aspirating a first volume/layer of liquid components. In some embodiments, this first volume/layer of liquid components may be made up of plasma that has been separated from blood. Then, according to the further method, the first volume of liquid components may be introduced into a second vessel (tube 12, tube 112 or bucket 212), which is loaded into a centrifuge for performance of a spinning process that separates the liquid material in the vessel into further separated liquid sub-components. For example, where the first volume of liquid components is made up of plasma, the plasma may be further separated into lower density platelet-poor plasma (PPP) in a top sub-component layer and higher density platelet-rich plasma (PRP) in a bottom sub-component layer. The diaphragm (14, 114, 214) may then be inserted into the vessel, and the top sub-component layer may be aspirated from the vessel, in a manner similar to that described above with respect to the first, second and third method embodiments. After the top sub-component layer has been aspirated, the diaphragm (14, 114, 214) may be removed from the vessel, and the remaining second sub-component layer in the bottom of the vessel (which may be below the level at which it is capable of being extracted through the diaphragm) may be removed by aspiration through a hollow pipette inserted into the vessel. The pipette may be made of plastic or metal, and in some embodiments, may have a blunt hollow distal end for insertion into the vessel, and a female luer-lock connector at an opposite end for connection to a syringe or similar device to facilitate aspiration of the second sub-component layer of liquid components from the vessel. In some embodiments, the pipette may have an inner diameter of 14 to 20 gauge, although in other embodiments the pipette may have a larger or smaller inner diameter.

### INTERPRETATION

In the various embodiments herein, it should be understood that the tube 12, the tube 112, and the bucket 212 may all be generally referred to as a “vessel” which may undergo centrifugation and from which liquid components may be aspirated, according to the description and techniques of this disclosure.

The term “approximately” means that something is almost, but not completely, accurate or exact; roughly. Additionally, the term “substantially” means to a great or significant extent; for the most part, essentially.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A system for aspirating liquid components from a vessel, the system comprising:
the vessel having a hollow inner portion, a sidewall radially extending around the hollow inner portion, an open end, and a closed end opposite the open end;
a diaphragm slideably coupled to the hollow inner portion of the vessel, wherein the diaphragm is configured to move towards the closed end and the open end, and wherein a hollow inner sub-portion located between the closed end and the diaphragm defines a first portion and a hollow inner sub-portion located between the diaphragm and the open end defines a second portion, and wherein the diaphragm comprises an aperture that is in fluid communication with the first portion.

2. The system of claim 1, further comprising:
a flexible pipe having a first open end and a second open end located opposite the first open end, wherein the first open end is coupled via an interface to the diaphragm, such that the flexible pipe is in fluid communication with the first portion, and wherein the flexible pipe extends from the diaphragm into the second portion and out of the vessel.

3. The system of claim 2, further comprising a syringe coupled to the second open end of the flexible pipe, wherein the syringe is configured to receive at least a portion of the liquid components through the flexible pipe.

4. The system of claim 2, wherein the diaphragm comprises a female luer attachment coupled to a top portion of the diaphragm, wherein the female luer attachment is configured to receive the first open end of the flexible pipe.

5. The system of claim 4, wherein the first open end of the flexible pipe comprises a male luer attachment configured to couple to the female luer attachment along the top portion of the diaphragm, and wherein the second open end of the flexible pipe comprises a female luer-valve attachment configured to couple to the syringe.

6. The system of claim 1, further comprising:
a seal coupled around a perimeter of the diaphragm, wherein the seal is configured to seal against the hollow inner portion to thereby prevent the liquid components from passing from the first portion to the second portion.

7. The system of claim 1, wherein the liquid components are separated liquid components, and wherein the vessel comprises a centrifuge tube or a centrifuge bucket.

8. The system of claim 1, wherein the vessel is composed of stainless steel.

9. The system of claim 1, wherein the diaphragm is composed of stainless steel.

10. The system of claim 1, wherein the closed end of the vessel defines a conical shape.

11. The system of claim 1, wherein the closed end of the vessel defines a flat bottom.

12. The system of claim 1, wherein a bottom side of the diaphragm defines a convex shape.

13. The system of claim 1, wherein a bottom side of the diaphragm defines a concave shape located within the convex shape.

14. The system of claim 1, wherein a bottom side of the diaphragm defines a conical shape having a peak at the aperture facing the open end of the vessel.

15. A method for aspirating liquid components from a vessel, the method comprising:
introducing liquid into the vessel having a hollow inner portion, a sidewall radially extending around the hollow inner portion, an open end, and a closed end opposite the open end, wherein the liquid comprises the liquid components;
separating the liquid into gradient layers within the vessel; and
inserting a diaphragm into the hollow inner portion, wherein a hollow inner sub-portion located between the closed end and the diaphragm defines a first portion and a hollow inner sub-portion located between the diaphragm and the open end defines a second portion; and
aspirating a first layer of liquid from the vessel through the diaphragm.

16. The method of claim 15, further comprising:
coupling a flexible pipe having a first open end and a second open end located opposite the first open end to the diaphragm such that the flexible pipe is in fluid communication with the first portion, wherein the flexible pipe extends from the diaphragm into the second portion and out of the vessel;
wherein aspirating the first layer of liquid from the vessel comprises aspirating the first layer of liquid through the diaphragm and the flexible pipe.

17. The method of claim 15, further comprising sealing the first portion from the second portion via a seal that extends around a perimeter of the diaphragm to thereby prevent the liquid components from passing from the first portion to the second portion.

18. The method of claim 15, wherein aspirating the first layer of liquid from the vessel comprises:
coupling a syringe to the diaphragm, either directly or through a flexible pipe; and
operating the syringe to draw the first layer of liquid into the syringe from the first portion of the vessel through the diaphragm.

19. The method of claim 15, wherein the method of aspirating liquid components from the vessel further comprises:
after aspirating the first layer of liquid from the vessel through the diaphragm, introducing the aspirated first layer of liquid into a separate, second vessel having a hollow inner portion, a sidewall radially extending around the hollow inner portion, an open end, and a closed end opposite the open end, wherein the first layer of liquid comprises liquid sub-components;
separating the first layer of liquid into gradient layers within the second vessel;
inserting a second diaphragm into the hollow inner portion of the second vessel, wherein a hollow inner sub-portion located between the closed end of the second vessel and the second diaphragm defines a third portion and a hollow inner sub-portion located between the second diaphragm and the open end of the second vessel defines a fourth portion; and
aspirating a second layer of liquid from the second vessel through the second diaphragm.

20. The method of claim 19, further comprising:
after aspirating the second layer of liquid from the second vessel through the second diaphragm, removing the second diaphragm from the second vessel, and aspirating a third layer of liquid remaining in the second vessel.
